(19) United States
(12) Patent Application Publication
(10) Pub. No.: US 2022/0372567 A1
(43) Pub. Date: Nov. 24, 2022

(54) MULTI-OMIC ANALYSIS OF EXTRACELLULAR VESICLES IN MONODISPERSE DROPLETS

(71) Applicant: Fluent Biosciences Inc., Watertown, MA (US)

(72) Inventors: Robert Meltzer, Belmont, MA (US); Catherine Kugler, Watertown, MA (US); Yi Xue, Shrewsbury, MA (US); Kristina Fontanez, Arlington, MA (US); Sepehr Kiani, Watertown, MA (US); Mehrtash Babadi, Watertown, MA (US)

(21) Appl. No.: 17/747,603

(22) Filed: May 18, 2022

Related U.S. Application Data
(60) Provisional application No. 63/190,079, filed on May 18, 2021.

(51) Int. Cl. C12Q 1/6869; C12Q 1/6806; C12N 15/10

(52) U.S. Cl. CPC C12Q 1/6869; C12Q 1/6806; C12N 15/1065; C12N 15/1096; C12Q 2563/159; C12Q 2563/179

(57) ABSTRACT

This disclosure provides methods and systems for single-extracellular (EV), multi-omic analysis of target EVs without microfluidic devices. The disclosed methods involve the use of template particles to template the formation of monodisperse droplets to generally capture a single target EV from a population of EVs in an encapsulation, derive a plurality of distinct mRNA molecules from the single target EV, and quantify the distinct mRNA molecules to generate an expression profile. Nucleic-acid-tagged antibody conjugates are used for simultaneous proteomic analysis along with the gene expression profiling, which enables classification of an EV in a sample.

### TECHNICAL FIELD

This disclosure relates to methods and systems for multi-omic profiling of extracellular vesicles.

### BACKGROUND

Extracellular vesicles (EVs) are small, lipid membrane-bound, subcellular compartments shed by cells. They can be found in nearly all biological fluids and are implicated in cell-to-cell communication and a variety of other pathophysiological processes. EVs are broadly divided into various subclasses, for example, exosomes, microvesicles, apoptotic bodies, oncosomes, and exomeres. Each subclass of EV differs by their cellular origin, biogenesis, size and characteristic biomarker composition, e.g., specific payloads of proteins and nucleic acids.

EVs represent an ideal target for non-invasive or minimally-invasive diagnostic methods for many distinct pathologies. Recently, EVs have been shown to carry genetic and proteomic signatures of their parent cells. These genetic and proteomic signatures are faithfully representative of their parent cells, as the lipid bilayer of the vesicles protects the nucleic acids and proteins in the EVs from degradation. Thus, for example, the otherwise-labile RNA transcriptome in an EV remains faithful to that of the parent cell that produced the EV. Similarly, the quantity of certain types of EV in blood has been shown useful in diagnosing or predicting certain disease states. For example, increased levels of apoptotic bodies have been linked to advanced disease states that feature increased cell death. Thus, given the distinguishable nature of EVs and their ability to protect biological targets representative of their parent cells, EVs are a prime candidate for use in diagnostic assays of pathologies, such as cancer.

EVs are present in biological fluids at high concentrations, from 0.88*10\hat{ }8 to 13.88*10\hat{ }8 EVs/mL. However, specific subclasses of EVs may present at far lower concentrations. Moreover, individual EVs carry a low information payload, e.g., nucleic acids and proteins, as compared to their parent cells. Accordingly, it is vital to be able to capture and sequence a large number of individual EVs, e.g., around 100,000 or more individual vesicles, in order to analyze a reasonable representation of vesicle content in a sample.

Unfortunately, current methods for assessing EVs limit their practical use. Currently, most clinical EV analysis is performed using bulk preparations, which average the effects of all EV classes and disease states. However, disease-derived EVs are often from rare or sparsely represented cell types, meaning that their signatures can be masked by those from EVs derived from healthy cells, which generally comprise the bulk of a sample.

Other methods for EV analysis require a number of time-intensive steps to isolate EVs from a sample. For example, characterizing an EV subtype generally involves roughly isolating EVs by size or density using physical separation methods and a subsequent analytical method, such as ELISA or Western blotting. This precludes their use for concurrently assessing the payload of an individual EV and determining the quantity of a particular type of EV in a sample.

### SUMMARY

This disclosure provides methods, compositions, and systems for multi-omic analyses of EVs in a sample, including individual EVs, without using microfluidics. The systems, methods, and compositions provide the ability to quickly characterize and quantify the subclasses of individual EVs in a sample, which can provide diagnostic information for a number of different pathologies. Further, not only can the classes of EVs be ascertained, but systems and methods of the invention also provide the ability to analyze and quantify components of individual EVs, e.g., nucleic acids and proteins. The results of such analyses can be used to provide new links between EVs and a number of health conditions.

Systems and methods of the invention operate by the generation of an emulsion with template particles to segregate individual target EVs into monodisperse droplets without the need for expensive and complicated microfluidics. Nucleic acid molecules are released from the target EVs inside the monodisperse droplets allowing them to be analyzed and quantified to, for example, generate an expression profile for individual EVs. Further, the EVs can be incubated with target-specific antibodies with conjugated oligonucleotide tags that permit proteomic analysis of the EVs. The EV-bound antibodies are segregated into the monodisperse droplets along with the individual EVs. In subsequent amplification and sequencing steps, the bound antibodies can be identified using barcode sequences in the nucleic acid tags. Identification and quantification of the bound antibodies provides qualitative and quantitative information on surface protein expression of an individual EV. In addition, the Inventors have found that proteins on the surface of EVs can be used to identify the subclass of an individual EV, which provides critical diagnostic information for a number of distinct pathologies. This approach provides a massively parallel analytical workflow that is inexpensive and scalable to ascertain multi-omic analysis of millions of individual EVs with a single library preparation.

Methods and systems of the invention use template particles to template the formation of monodisperse droplets and isolate target EVs for profiling. An exemplary method of the invention for analyzing EVs in a sample includes, creating an aqueous mixture that includes EVs, target-specific antibodies linked to index oligonucleotides, and template particles decorated with capture oligonucleotides. A partitioning oil is added to the aqueous mixture. The mixture is then sheared to simultaneously form a plurality of water-in-oil partitions. Each EV is isolated in one of the partitions with one of the template particles and is bound to at least one of the antibodies. Each EV in a partition is lysed, which releases a nucleic acid from the EV. Then, the index oligonucleotides from the antibody bound to the EV and the released nucleic acid are captured.

In certain aspects, the index oligonucleotides include a barcode sequence that identifies a protein to which the antibody binds. The capture oligonucleotides of each template particle may also comprise a partition barcode unique to each template particle. This can be used to identify the partition/template in which a particular index oligonucleotide and/or released nucleic acid was captured.

Methods of the invention may further include creating a sequencing library containing copies of the index oligonucleotide barcodes, the partition barcode, and/or the released nucleic acid. The sequencing library can be sequenced to produce sequence reads. The sequence reads are used, for example, to identify proteins and nucleic acids present in the EVs. In certain aspects, this may include using the partition barcodes in the sequence reads to identify proteins and nucleic acids of at least one individual EV.

Certain methods also include using the index oligonucleotides and/or the released nucleic acid to identify an extracellular vesicle subclass of the individual EVs. The identified EV subclasses include, for example, an exosome, a microvesicle, an apoptotic body, an oncosome, and an exomere.

In certain methods, the released nucleic acid is RNA. Thus, methods can also include reverse transcribing the released RNA captured by the capture oligonucleotides to produce a cDNA library. The released RNA can include, for example, one or more of mRNA, microRNA, ncRNA, tRNA, snRNA, and vault RNA. In certain methods, the RNA is mRNA.

The EVs in the aqueous mixture typically are obtained from a biological sample. Such methods may further include assessing a pathology in the subject using the identified extracellular vesicle subclass of one or more individual EVs in the sample. The methods may also include quantifying amounts of individual EVs in the sample of a particular extracellular vesicle subclass. The released nucleic acids and/or proteins identified in the EVs may also be analyzed to assess the pathology.

In certain methods, the target-specific antibodies are part of a panel of target-specific antibodies, which each bind to a different protein. In exemplary panels, at least one antibody specifically binds to a protein selected from CD63, CD9, C3b TSP, Annexin V, Phosphatidylserine, CD40L, an integrin, and ARF6. In an exemplary method, the pathology is cancer and the extracellular vesicle subclass is an oncosome.

### BRIEF DESCRIPTION OF DRAWINGS

FIG. 1 diagrams a method for single EV profiling.

FIG. 2 illustrates a droplet according to one aspect of the invention.

FIG. 3 illustrates a droplet following lysis of a target EV.

FIG. 4 illustrates the capture of mRNA.

FIG. 5 illustrates synthesis of cDNA to form a first strand.

FIG. 6 illustrates amplification of a first strand to generate an amplicon.

FIG. 7 illustrates a method for sequence-specific capture of mRNA.

FIG. 8 illustrates synthesis of cDNA to form a first strand.

FIG. 9 illustrates amplification of a first strand to generate an amplicon.

FIG. 10 shows an exemplary nucleic-acid-labelled antibody conjugate.

FIG. 11 illustrates the capture of mRNA according to TSO embodiments.

FIG. 12 shows a first strand following TSO-PCR amplification.

FIG. 13 shows selected types of extracellular vesicle.

FIGS. 14A-14F show an exemplary method of the disclosure.

FIG. 15 provides a table of select EV-specific target proteins.

### DETAILED DESCRIPTION

This disclosure provides systems and methods of using template particles to form monodisperse droplets for segregating individual extracellular vesicles (EV) in fluid partitions for multi-omic analyses that can, for example, be used to identify and quantify the various subclasses of EVs in a sample.

The disclosed systems and methods involve the use of template particles to template the formation of monodisperse droplets to generally capture a single EV and template particle in a water-in-oil partition. The EV in a particular partition can be identified, for example, using a target-specific antibody that binds to a certain protein on the surface of the EV. The antibody may be attached to an index oligonucleotide that identifies the antibody, and by extension, the protein to which it binds. The index oligonucleotides can be captured by the template particle in the partition using, for example, capture oligonucleotides on the surface of the particle. Similarly, the EV in a partition can be lysed to release biological components from the EV, such as one or more nucleic acids. The biological components can be captured by capture oligonucleotides on the template particle for analysis.

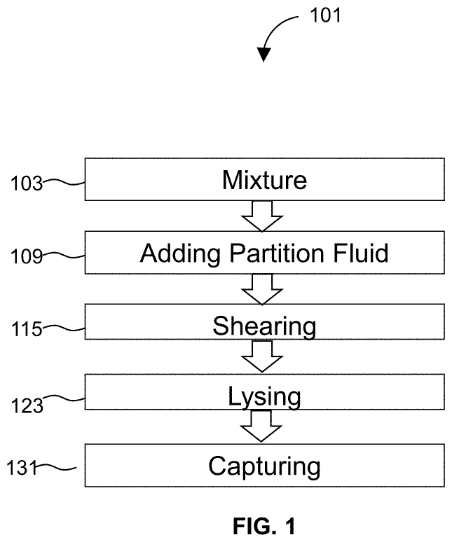

FIG. 1 diagrams a method 101 for analyzing EVs in a sample. The method 101 includes preparing an aqueous mixture 103 that includes one or more target EVs, target-specific antibodies, and template particles that are each linked to an index oligonucleotide. The aqueous mixture can also include additional components, e.g., buffers and reagents. In the aqueous mixture, the target-specific antibodies bind to cognate antigens, target proteins on the surface of a target EV.

In certain aspects, the EVs may be washed to remove unbound antibody conjugates before combining the EVs with the template particles in the aqueous mixture. While any suitable order may be used, in some instances, a tube may be provided comprising the template particles. The tube can be any type of tube, such as a sample preparation tube sold under the trade name Eppendorf, or a blood collection tube, sold under the trade name Vacutainer. Template particles may be in dried format. Preparing the aqueous mixture 103 may include using a pipette to pipette a sample comprising EVs and, for example, an aqueous fluid into the tube containing template particles.

After the aqueous mixture is prepared 103, a partitioning fluid is added 109 to the aqueous mixture. The portioning fluid is a fluid, e.g., an oil, that is immiscible with the aqueous mixture.

The method 101 then includes shearing 115 the mixture and partitioning fluid to generate monodisperse water-in-oil droplets, i.e., partitions. Preferably, the shearing step includes agitating the tube containing the fluids using a vortexer or any method of controlled or uncontrolled agitation, such as shaking, pipetting, pumping, tapping, sonication and the like. After agitating (e.g., vortexing/shearing 115), a plurality (e.g., thousands, tens of thousands, hundreds of thousands, one million, two million, ten million, or more) of aqueous partitions are formed essentially simultaneously. The vortexing/shearing step causes the fluids to partition into a plurality of monodisperse droplets. A substantial portion of droplets will contain a single template particle and a single target EV bound to one or more antibody conjugates. Droplets containing more than one or none of a template particle or target EV can be removed, destroyed, or otherwise ignored.

The next step of the method 101 is to lyse 123 the target EVs. EV lysis 123 may be induced by a stimulus, such as, for example, lytic reagents, detergents, or enzymes. Reagents to induce EV lysis may be provided by the template particles via internal compartments or a porous structure in the hydrogel of the template particles. In certain aspects, the lysing step 123 involves heating the monodisperse droplets to a temperature sufficient to release lytic reagents contained inside the template particles into the monodisperse droplets. This accomplishes EV lysis 123 of the target EVs, thereby releasing biological components, e.g., nucleic acids, inside of the droplets that contained the target EVs.

After lysing 123 target EVs inside the droplets, one or more released biological components are captured 131 by a capture moiety on the template particle. In exemplary methods, the released components include at least one nucleic acid, which is captured 131 by a capture oligonucleotide on the template particle. Similarly, the index oligonucleotide from the antibody conjugate is captured 131 by a capture oligonucleotide on the template particle.

In an exemplary method, the released biological components include one or more mRNA. The released mRNA is captured 131 by a capture oligonucleotide on the template particle, reverse transcribed and, along with the nucleic acid labels of EV-protein-bound antibody conjugates, amplified and analyzed by, for example, nucleic acid sequencing.

In order to sequence and quantify mRNA, reverse transcription is carried out to generate a library including cDNA with a barcode sequence that allows each library sequence to be traced back to the single EV from which the mRNA was derived. In preferred methods, template particles isolated with the mRNA include a plurality of barcoded capture sequences that hybridize with target mRNA. After hybridization, cDNA is synthesized by reverse transcription. Reagents for reverse transcription can be provided in a variety of ways in a variety of formats. In some instances, reagents and reverse transcriptase are provided by the template particles. Once a library is generated comprising barcoded cDNA, the cDNA can be amplified, by for example, PCR, to generate amplicons for sequencing.

The index oligonucleotides of the antibody conjugates can include a PCR handle that functions as a primer site used for subsequent PCR amplification. Accordingly, the inclusion of PCR-handle-specific primers during amplification of the barcoded cDNA library will result in amplification of both mRNA-derived cDNA and antibody-conjugate index oligonucleotides for subsequent sequencing. In exemplary methods, the index oligonucleotides include a poly A tag or other sequence complementary to the plurality of barcoded capture sequences present in or on the template particles. Inclusion of a poly A tag allows for the use of poly T barcoded capture sequences to hybridize both the antibody index oligonucleotides and mRNA from the lysed EV for gene expression profiling. Primer domains for subsequent PCR amplification can then be introduced to antibody tags as part of the capture sequence barcode that hybridize with target mRNA. Sequence reads are processed according to methods described herein to accomplish the quantification of mRNA and protein expression.

In some aspects, the target EVs include EVs obtained from, for example, a sample (tissue of bodily fluid) of a patient. The sample may include a fine needle aspirate, a biopsy, or a bodily fluid from the patient. Upon being isolated from the sample, the EVs may be processed by, for example, generating a suspension with an appropriate solution. Such solution will generally be a balanced salt solution, e.g. normal saline, PBS, Hank's balanced salt solution, etc., and in certain instances supplemented with fetal calf serum or other naturally occurring factors, in conjunction with an acceptable buffer at low concentration, generally from 5-25 mM. Convenient buffers include HEPES, phosphate buffers, lactate buffers, etc. The separated EVs can be collected in any appropriate medium that maintains the viability of the EVs, usually having a cushion of serum at the bottom of the collection tube. Various media are commercially available and may be used according to the nature of the EVs, including dMEM, HBSS, dPBS, RPMI, Iscove's medium, etc., frequently supplemented with fetal calf serum.

Methods and systems of the invention use template particles to template the formation of monodisperse droplets and isolate single target EVs. The disclosed template particles and methods for targeted library preparation thereof leverage the particle-templated emulsification technology previously described in, Hatori et. al., Anal. Chem., 2018 (90):9813-9820, which is incorporated by reference. Essentially, micron-scale beads (such as hydrogels) or “template particles” are used to define an isolated fluid volume surrounded by an immiscible partitioning fluid and stabilized by temperature insensitive surfactants.

The template particles of the present disclosure may be prepared using any method known in the art. Generally, the template particles are prepared by combining hydrogel material, e.g., agarose, alginate, a polyethylene glycol (PEG), a polyacrylamide (PAA), Acrylate, Acrylamide/bisacrylamide copolymer matrix, and combinations thereof. Following the formation of the template particles they are sized to the desired diameter. In some embodiments, sizing of the template particles is done by microfluidic co-flow into an immiscible oil phase.

In some embodiments of the template particles, a variation in diameter or largest dimension of the template particles such that at least 50% or more, e.g., 60% or more, 70% or more, 80% or more, 90% or more, 95% or more, or 99% or more of the template particles vary in diameter or largest dimension by less than a factor of 10, e.g., less than a factor of 5, less than a factor of 4, less than a factor of 3, less than a factor of 2, less than a factor of 1.5, less than a factor of 1.4, less than a factor of 1.3, less than a factor of 1.2, less than a factor of 1.1, less than a factor of 1.05, or less than a factor of 1.01.

Template particles may be porous or nonporous. In any suitable embodiment herein, template particles may include microcompartments (also referred to herein as “internal compartments”), which may contain additional components and/or reagents, e.g., additional components and/or reagents that may be releasable into monodisperse droplets as described herein. Template particles may include a polymer, e.g., a hydrogel. Template particles generally range from about 0.1 to about 1000 μm in diameter or larger dimension. In some embodiments, template particles have a diameter or largest dimension of about 1.0 μm to 1000 μm, inclusive, such as 1.0 μm to 750 μm, 1.0 μm to 500 μm, 1.0 μm to 250 μm, 1.0 μm to 200 μm, 1.0 μm to 150 μm 1.0 μm to 100 μm, 1.0 μm to 10 μm, or 1.0 μm to 5 μm, inclusive. In some embodiments, template particles have a diameter or largest dimension of about 10 μm to about 200 μm, e.g., about 10 μm to about 150 μm, about 10 μm to about 125 μm, or about 10 μm to about 100 μm.

In practicing the methods as described herein, the composition and nature of the template particles may vary. For instance, in certain aspects, the template particles may be microgel particles that are micron-scale spheres of gel matrix. In some embodiments, the microgels are composed of a hydrophilic polymer that is soluble in water, including alginate or agarose. In other embodiments, the microgels are composed of a lipophilic microgel.

In other aspects, the template particles may be a hydrogel. In certain embodiments, the hydrogel is selected from naturally derived materials, synthetically derived materials and combinations thereof. Examples of hydrogels include, but are not limited to, collagen, hyaluronan, chitosan, fibrin, gelatin, alginate, agarose, chondroitin sulfate, polyacrylamide, polyethylene glycol (PEG), polyvinyl alcohol (PVA), acrylamide/bisacrylamide copolymer matrix, polyacrylamide /poly(acrylic acid) (PAA), hydroxyethyl methacrylate (HEMA), poly N-isopropylacrylamide (NIPAM), and polyanhydrides, poly(propylene fumarate) (PPF).

In some aspects, the presently disclosed template particles further comprise materials which provide the template particles with a positive surface charge, or an increased positive surface charge. Such materials may be without limitation poly-lysine or Polyethyleneimine, or combinations thereof. This may increase the chances of association between the template particle and, for example, an EV which generally have a mostly negatively charged membrane.

Other strategies may be used to increase the chances of template particle-target EV association, which include creation of specific template particle geometry. For example, in some embodiments, the template particles may have a general spherical shape, but the shape may contain features such as flat surfaces, craters, grooves, protrusions, and other irregularities in the spherical shape.

Any one of the above described strategies and methods, or combinations thereof may be used in the practice of the presently disclosed template particles and method for targeted library preparation thereof. Methods for generation of template particles, and template particles-based encapsulations, were described in International Patent Publication WO 2019/139650, which is incorporated herein by reference.

Creating template particle-based encapsulations for single EV expression profiling comprises combining target EVs with a plurality of template particles in a first fluid to provide a mixture in a reaction tube. The mixture may be incubated to allow association of the plurality of the template particles with target EVs. A portion of the plurality of template particles may become associated with the target EVs. The mixture is then combined with a second fluid which is immiscible with the first fluid. The fluid and the mixture are then sheared so that a plurality of monodisperse droplets is generated within the reaction tube. The monodisperse droplets generated comprise (i) at least a portion of the mixture, (ii) a single template particle, and (iii) a single target EV. Of note, in practicing methods of the invention provided by this disclosure a substantial number of the monodisperse droplets generated will comprise a single template particle and a single target EV, however, in some instances, a portion of the monodisperse droplets may comprise none or more than one template particle or target EV.

In certain aspects, to increase the chances of generating an encapsulation, such as, a monodisperse droplet that contains one template particle and one target EV, the template particles and target EVs are combined at a ratio wherein there are more template particles than target EVs. For example, the ratio of template particles to target EVs combined in a mixture as described above may be in a range of 5:1 to 1,000:1, respectively. In other embodiments, the template particles and target EVs are combined at a ratio of 10:1, respectively. In other embodiments, the template particles and target EVs are combined at a ratio of 100:1, respectively. In other embodiments, the template particles and target EVs are combined at a ratio of 1000:1, respectively.

To generate a monodisperse emulsion, the presently disclosed method includes a step of shearing/vortexing 115 the second mixture provided by combining a first mixture comprising template particles and target EVs with a second fluid immiscible with the first mixture. Any suitable method or technique may be utilized to apply a sufficient shear force to the second mixture. For example, the second mixture may be sheared by flowing the second mixture through a pipette tip. Other methods include, but are not limited to, shaking the second mixture with a homogenizer (e.g., vortexer), or shaking the second mixture with a bead beater. In some embodiments, vortexing may be performed for example for 15 seconds, or in the range of 15 seconds to 5 minutes. The application of a sufficient shear force breaks the second mixture into monodisperse droplets that encapsulate one of a plurality of template particles and a target EV.

In some aspects, generating the template particles-based monodisperse droplets involves shearing two liquid phases. The mixture is the aqueous phase and, in some embodiments, comprises reagents selected from, for example, buffers, salts, lytic enzymes (e.g. proteinase k) and/or other lytic reagents (e. g. Triton X-100, Tween-20, IGEPAL, bm 135, or combinations thereof), nucleic acid synthesis reagents e.g. nucleic acid amplification reagents or reverse transcription mix, or combinations thereof. The fluid is the continuous phase and may be an immiscible oil such as fluorocarbon oil, a silicone oil, or a hydrocarbon oil, or a combination thereof. In some embodiments, the fluid may comprise reagents such as surfactants (e.g. octylphenol ethoxylate and/or octylphenoxypolyethoxyethanol), reducing agents (e.g. DTT, beta mercaptoethanol, or combinations thereof).

In practicing the methods as described herein, the composition and nature of the monodisperse droplets, e.g., single-emulsion and multiple-emulsion droplets, may vary. As mentioned above, in certain aspects, a surfactant may be used to stabilize the droplets. The monodisperse droplets described herein may be prepared as emulsions, e.g., as an aqueous phase fluid dispersed in an immiscible phase carrier fluid (e.g., a fluorocarbon oil, silicone oil, or a hydrocarbon oil) or vice versa. Accordingly, a droplet may involve a surfactant stabilized emulsion, e.g., a surfactant stabilized single emulsion or a surfactant stabilized double emulsion. Any convenient surfactant that allows for the desired reactions to be performed in the droplets may be used. In other aspects, monodisperse droplets are not stabilized by surfactants.

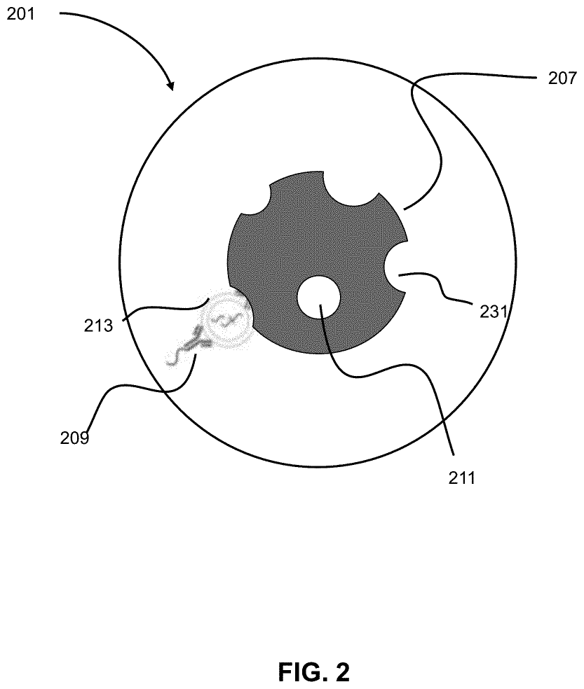

FIG. 2 illustrates a droplet 201 according to one aspect of the invention. The depicted droplet 201 is a single one of a plurality of monodisperse droplets generated by shearing a mixture according to methods of the invention. The droplet 201 includes a template particle 207 and a single target EV 213. In certain aspects, the template particle 207 has crater-like depressions 231 to facilitate capture of single EVs 213. The template particle 207 may also include an internal compartment 211 to deliver one or more reagents into the droplet 201 upon stimulus. The target EV 213 may have optionally been exposed to nucleic-acid-labelled antibody conjugates prior to droplet formation. After washing away unbound antibody conjugates, the EV will carry along any bound antibody conjugates into the droplet 201 such that subsequent sequencing data showing the presence of one or more index oligonucleotide tags is indicative of the expression of that antibody conjugate's target protein by the target EV 213. Accordingly, a single amplification and sequencing reaction can provide quantitative and qualitative information regarding gene expression through mRNA analysis as well as protein expression data.

In certain methods, the template particles contain multiple internal accessible volumes. The internal volumes of the template particles may be used to encapsulate reagents that can be triggered to release a desired compound, e.g., a substrate for an enzymatic reaction, or induce a certain result, e.g. lysis of an associated target EV. Reagents encapsulated in the template particles' compartment may be without limitation reagents selected from buffers, salts, lytic enzymes (e.g. proteinase k), other lytic reagents (e. g. Triton X-100, Tween-20, IGEPAL, bm 135), nucleic acid synthesis reagents, or combinations thereof. The internal volumes may be micron scale structural features in the hydrogel of a template particle. Alternatively or additionally, an internal volume may be defined by the hydrogel mesh of the template particle. In certain aspects, the hydrogel has a mesh length less than 200 nm.

Lysis of single target EVs occurs within the monodisperse droplets and may be induced by a stimulus such as heat, osmotic pressure, lytic reagents (e.g., DTT, beta-mercaptoethanol), detergents (e.g., SDS, Triton X-100, Tween-20), enzymes (e.g., proteinase K), or combinations thereof. In some embodiments, one or more of the said reagents (e.g., lytic reagents, detergents, enzymes) is compartmentalized within the template particle. In other embodiments, one or more of the said reagents is present in the mixture. In some other embodiments, one or more of the said reagents is added to the solution comprising the monodisperse droplets, as desired.

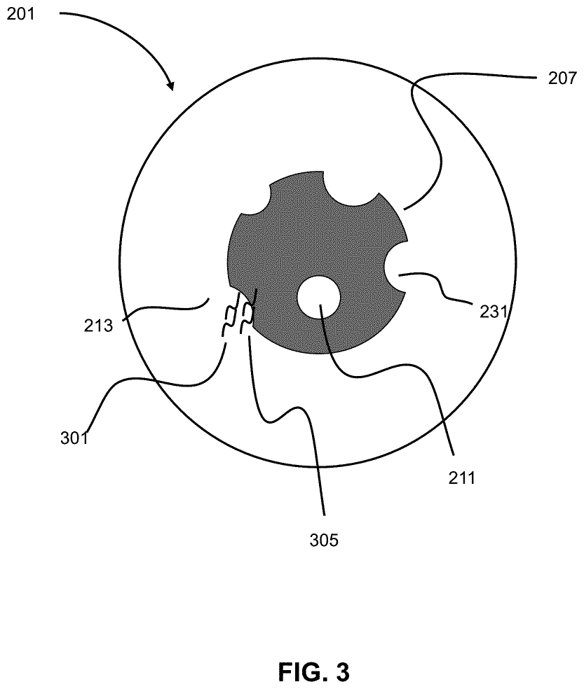

FIG. 3 illustrates a droplet 201 following lysis of a target EV. The depicted droplet 201 includes a template particle 207 and released mRNA 301 and index oligonucleotide tags 305 from antibody conjugates that had bound target proteins on the lysed target EV. Methods of the invention quantify amplified products of the released mRNAs 301 and index oligonucleotide tags 305, preferably by sequencing.

In preferred embodiments, template particles comprise a plurality of capture probes. Generally, the capture probe of the present disclosure is an oligonucleotide. In certain aspects, the capture probes are attached to the template particle's material, e.g. hydrogel material, via covalent acrylic linkages. In some aspects, the capture probes are acrydite-modified on their 5′ end (linker region). Generally, acrydite-modified oligonucleotides can be incorporated, stoichiometrically, into hydrogels such as polyacrylamide, using standard free radical polymerization chemistry, where the double bond in the acrydite group reacts with other activated double bond containing compounds such as acrylamide. Specifically, copolymerization of the acrydite-modified capture probes with acrylamide including a crosslinker, e.g. N,N′-methylenebis, will result in a crosslinked gel material comprising covalently attached capture probes. In some other aspects, the capture probes comprise an Acrydite terminated hydrocarbon linker and combining the said capture probes with a template particle will cause their attachment to the template particle.

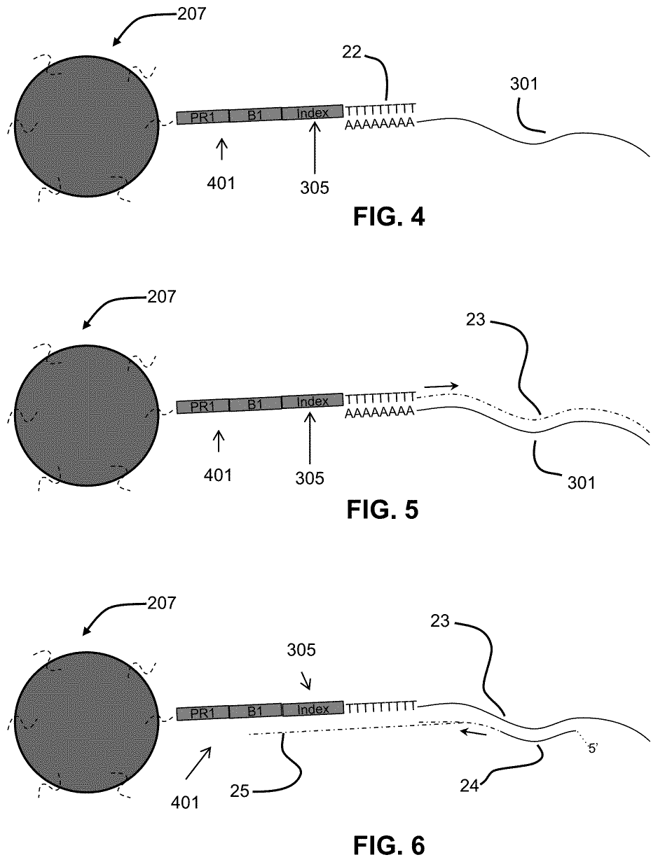

FIGS. 4-6 show an exemplary method for nonspecific amplification of mRNA according to certain aspects of the disclosure. In particular, the method relies on the presence of the poly A tail at the 3′ end of a mRNA for the non-specific capture of mRNAs. A poly A sequence may be included in the index oligonucleotides of the antibody conjugates so that the same capture probes can capture both target mRNA and target antibody labels.

FIG. 4 illustrates the capture of mRNA 301 but can be similarly applied to the capture of target antibody index oligonucleotides and/or other released biological components, e.g., proteins and other nucleic acids, which can occur simultaneously for multi-omic analysis. Shown, is a template particle 207 comprising a plurality of capture probes 401 illustrated schematically by curved broken lines. One of the capture probes 401 is featured in a larger scale and in detail. The capture probe 401 preferably comprises, from 5′ end to 3′ end, a linker region to allow covalent bond with the template particle 207, a PR1 nucleotide sequence region comprising a universal primer nucleotide sequence, at least one barcode region B1, which may include an index nucleotide sequence index, and/or a UMI. In certain aspects, the capture probe 401 further includes a capture nucleotide sequence 22 comprising a poly T nucleotide sequence. A released nucleic acid, i.e., mRNA molecule 301 comprising a poly A sequence attaches to the capture probe's poly T sequence 22 via complementary base pairing. Following the hybridization of the mRNA molecule 301 and the capture probe 401, a reverse transcriptase is used to perform a reverse transcription reaction to synthesize cDNA and thereby create a first strand comprising the cDNA and the capture probe sequence. Index oligonucleotide tags 305 from the antibody conjugates will be similarly captured due to the inclusion of a poly A sequence and, in the case of an RNA nucleic acid tag, can undergo reverse transcription along with the captured mRNA 301 from the target EV. In the case of DNA index oligonucleotide tag 305, the tags will not undergo reverse transcription and will simply remain bound to the template particle 207 via a capture probe 401 and await subsequent amplification along with cDNA synthesized from the captured mRNA 301.

FIG. 5 illustrates synthesis of cDNA to form a first strand 23. A reverse transcriptase (not shown) synthesizes cDNA from mRNA that is hybridized to a poly T sequence of a capture probe 401. After synthesis, a first strand 23 is formed, wherein the first strand 23 comprises the cDNA and the capture probe 401 sequence. Following synthesis, the mRNA molecule 301 and first strand 23 hybrid may be denatured (not shown) using any method traditional in the art, such as an exposure to a denaturing temperature.

FIG. 6 illustrates amplification of a first strand 23 to generate an amplicon. In particular, following the formation of a first strand 23, a second strand primer 24 comprising a random sequence, such as, a random hexamer, anneals with the first strand 23 to form a DNA-primer hybrid. A DNA polymerase is used to synthesize a complementary second strand 25, i.e., an amplicon. In the embodiment illustrated, the second strand primer 24 comprises a “tail” region which does not hybridize with the first strand 23. In some embodiments, the tail region comprises a second universal primer sequence. The second strand 25 may be further amplified by PCR to generate a plurality of amplicons, and quantified by DNA sequencing. Similar universal primer sequences can be included in index oligonucleotide tags from the antibody conjugates such that those tags will be simultaneously amplified using the same primers as the mRNA-derived cDNA.

Amplification or nucleic acid synthesis, as used herein, generally refers to methods for creating copies of nucleic acids by using thermal cycling to expose reactants to repeated cycles of heating and cooling, and to permit different temperature-dependent reactions (e.g. by polymerase chain reaction (PCR). Any suitable PCR method known in the art may be used in connection with the presently described methods. Non limiting examples of PCR reactions include real-time PCR, nested PCR, multiplex PCR, quantitative PCR, TS-PCR, or touchdown PCR.

The terms “nucleic acid amplification reagents” or “reverse transcription mix” encompass without limitation dNTPs (mix of the nucleotides dATP, dCTP, dGTP and dTTP), buffer/s, detergent/s, or solvent/s, as required, and suitable enzyme such as polymerase or reverse transcriptase. The polymerase used in the presently disclosed targeted library preparation method may be a DNA polymerase, and may be selected from, but is not limited to, Taq DNA polymerase, Phusion polymerase, or Q5 polymerase. The reverse transcriptase used in the presently disclosed targeted library preparation method may be for example, Moloney murine leukemia virus (MMLV) reverse transcriptase, or maxima reverse transcriptase. In some embodiments, the general parameters of the reverse transcription reaction comprise an incubation of about 15 minutes at 25 degrees and a subsequent incubation of about 90 minutes at 52 degrees. Nucleic acid amplification reagents are commercially available, and may be purchased from, for example, New England Biolabs, Ipswich, Mass., USA, or Clonetech.

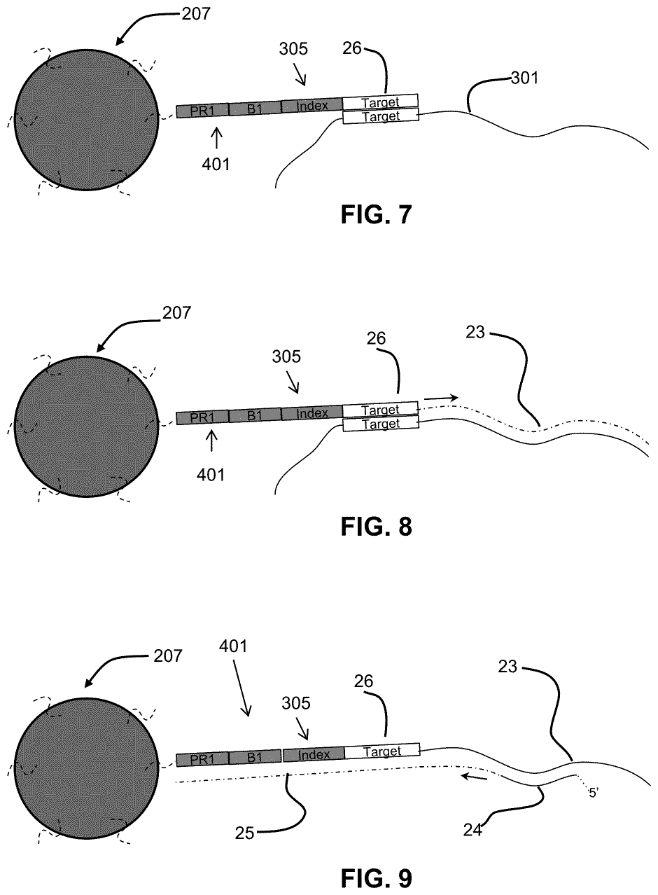

FIGS. 7-9 illustrate a method for sequence-specific amplification of mRNA according to certain aspects of the disclosure but can be similarly applied to the capture of target antibody index oligonucleotide tags, and/or other biological components released from a partitioned target EV, which can occur simultaneously for multi-omic analysis.

FIG. 7 illustrates a method for sequence-specific capture of mRNA 301. The template particle 207 comprises a plurality of capture probes 401 illustrated schematically by curved broken lines. A featured capture probe 401 comprises, from 5′ end to 3′ end, a linker region to allow covalent bond with the template particle 207, a PR1 region comprising a universal primer nucleotide sequence, at least one barcode region B1, which may include an index sequence 305, and/or a UMI. As shown, the capture probe 401 may further include a capture sequence comprising a gene-specific sequence 26. Capture probes 401 can be included wherein the gene-specific sequence 26 is substituted with various complementary sequences to barcodes or tags included in the index oligonucleotide tags of the antibody conjugates.

By using separate capture sequences, competition for binding between mRNA and antibody index oligonucleotides can be avoided along with resulting bias in the data. A molecule of mRNA 301, released inside a monodisperse droplet, comprising a sequence complementary to the gene-specific sequence 26 attaches to the capture probe's gene-specific sequence 26 via complementary base pairing. The gene-specific or transcript-specific sequence may include any sequence of interest, for example, a sequence corresponding to an oncogene or associated with a particular EV subclass.

In certain aspects, template particles 207 according to aspects of the invention may include capture probes with certain sequences specific to genes of interest, such as, oncogenes. Some non-limiting examples of genes of interest that may be assayed for include, but are not limited to, BAX, BCL2L1, CASP8, CDK4, ELK1, ETS1, HGF, JAK2, JUNB, JUND, KIT, KITLG, MCL1, MET, MOS, MYB, NFKBIA, EGFR, Myc, EpCAM, NRAS, PIK3CA, PML, PRKCA, RAF1, RARA, REL, ROS1, RUNX1, SRC, STAT3, CD45, cytokeratins, CEA, CD133, HER2, CD44, CD49f, CD146, MUC1/2, ABL1, AKT1, APC, ATM, BRAF, CDH1, CDKN2A, CTNNB1, EGFR, ERBB2, ERBB4, EZH2, FBXW7, FGFR2, FGFR3, FLT3, GNAS, GNAQ, GNA11, HNF1A, HRAS, IDH1, IDH2, JAK2, JAK3, KDR, KIT, KRAS, MET, MLH1, NOTCH1, NPM1, NRAS, PDGFRA, PIK3CA, PTEN, PTPN11, RB1, RET, SMAD4, STK11, TP53, VHL, and ZHX2.

FIG. 8 illustrates the synthesis of cDNA to form a first strand 23. A reverse transcriptase (not shown) synthesizes cDNA from mRNA that is hybridized to a gene-specific sequence 26 of a capture probe 401 Following the hybridization of the target mRNA molecule 301 and the capture probe 401, a reverse transcription reaction is performed to synthesize cDNA and create a first strand 23. The first strand 23 comprises synthesized cDNA and the capture probe 401 sequence. The target mRNA molecule-first strand hybrid is then denatured using methods traditional in the art (not shown), and second strand primer comprising a random hexamer sequence anneals with complementary sequence of the first strand 23 to form a DNA-primer hybrid.

FIG. 9 illustrates amplification of a first strand 23 to generate an amplicon 25. In particular, following the formation of a first strand 23, a second strand primer 24 comprising a random sequence, such as, a random hexamer, anneals with the first strand 23 to form a DNA-primer hybrid. A DNA polymerase is used to synthesize a complementary second strand 25, i.e., an amplicon 25. In the embodiment illustrated, the second strand primer 24 comprises a “tail” region which does not hybridize with the first strand 23. In some embodiments, the tail region comprises a second universal primer sequence.

According to aspects of the present disclosure, the term “universal primer sequence” generally refers to a primer binding site, e.g., a primer sequence that would be expected to hybridize (base-pair) to, and prime, one or more loci of complementary sequence, if present, on any nucleic acid fragment. In some embodiments, the universal primer sequences used with respect to the present methods are P5 and P7.

The term barcode region may comprise any number of barcodes, index or index sequence, UMIs, which are unique, i.e., distinguishable from other barcode, or index, UMI sequences. The sequences may be of any suitable length which is sufficient to distinguish the barcode, or index, sequence from other barcode sequences. A barcode, or index, sequence may have a length of 4, 5, 6, 7, 8, 9, 10, 11, 12, 13, 14, 15, 16, 17, 18, 19, 20, 21, 22, 23, 24, 25 nucleotides, or more. In some embodiments, the barcodes, or indices, are predefined or selected at random.

In some methods of the invention, a barcode sequence may comprise unique molecular identifiers (UMIs). UMIs are a type of barcode that may be provided to a sample to make each nucleic acid molecule, together with its barcode, unique, or nearly unique. This may be accomplished by adding one or more UMIs to one or more capture probes of the present invention. By selecting an appropriate number of UMIs, every nucleic acid molecule in the sample, together with its UMI, will be unique or nearly unique.

UMIs are advantageous in that they can be used to correct for errors created during amplification, such as amplification bias or incorrect base pairing during amplification. For example, when using UMIs, because every nucleic acid molecule in a sample together with its UMI or UMIs is unique or nearly unique, after amplification and sequencing, molecules with identical sequences may be considered to refer to the same starting nucleic acid molecule, thereby reducing amplification bias. Methods for error correction using UMIs are described in Karlsson et al., 2016, “Counting Molecules in cell-free DNA and single cells RNA”, Karolinska Institute, Stockholm Sweden, incorporated herein by reference.

Such UMIs, present in the nucleic acid tags of antibody conjugates according to the invention, can allow for relative quantification of various expressions of proteins by the target EV by permitting the grouping of antibody tag amplicons by molecule of origin.

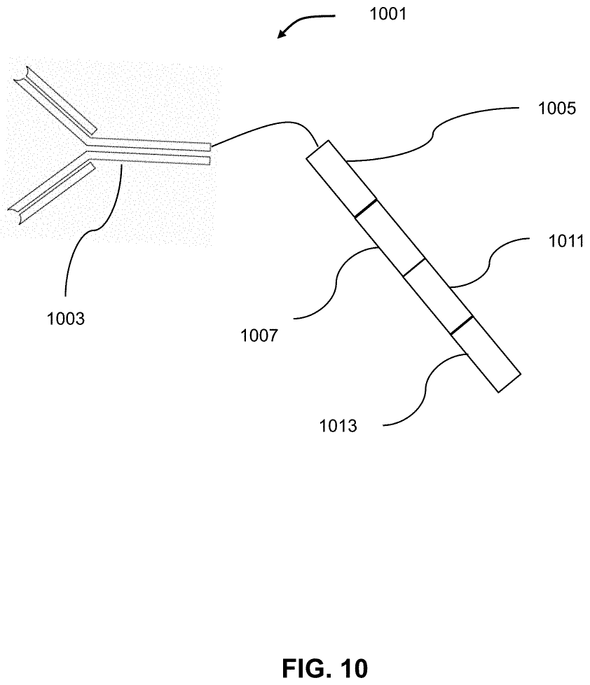

For proteomic analysis, EV samples can be incubated with a mixture comprising one or more index oligonucleotide labeled antibody conjugates. An exemplary antibody conjugate is shown in FIG. 10. Additional antibody conjugates and processes for their use in multi-omic analysis along with mRNA gene expression profiling can be found in descriptions of CITE-seq including Stoeckius, et al., 2017, Simultaneous epitope and transcriptome measurement in single cells, Nature Methods, 14:865-868.

As shown in FIG. 10, labelled antibody conjugates 1001 may include an antibody 1003 which can be selected based on the target protein and/or EV to be analyzed. For example, where expression of a certain EV surface protein, or lack thereof, is indicative of a EV subclass or certain disease, antibodies 1003 that specifically bind that surface protein may be used.

Linked to the antibody 1003 is an index oligonucleotide tag or label that may comprise various sequence portions. For example, as shown in FIG. 10, the index oligonucleotide tag or label may include a PCR handle 1005 or universal primer site used for subsequent PCR amplification as discussed above. The nucleic acid tag or label may include a barcode 1007 that is specific to the antibody 1003 to which it is linked and can be used to subsequently identify the antibody 1003 in by sequence analysis.

Additional components may include a UMI 1011 which can be used where multiple copies of a single type of antibody conjugate 1001 are used in order to collapse sequencing reads and remove amplification or sequencing or errors in quantifying protein expression. The tag may also include a capture portion 1013 that is complementary to the capture sequence on template particles to allow capture of the tags 1001 for subsequent amplification and the potential addition of further adapter sequences in a similar fashion as described with respect to the mRNA methods above. In preferred methods and systems, the capture portion comprises a poly A sequence to allow poly T capture probes to be used to hybridize both mRNA and antibody conjugate tags for multi-omic analysis.

Incubation of target EVs with the index oligonucleotide labeled antibody conjugates can occur in a buffer that promotes EV viability and reliable antibody conjugation. EVs may be washed post-incubation to remove any unbound antibody conjugates. The antibody labeled EVs can then be put in suspension with template particles and separated into monodisperse droplets as described above for EV capture, lysis, and mRNA hybridization as described above.

At this stage, antibody tags will be captured by their appropriate capture probes alongside mRNA from the lysed target EV. Emulsions can then be broken, the templates washed, and cDNA generated by reverse transcription. The cDNA can then be amplified which should generate the profile of captured cDNA as described but should also generate a significant population of short sequences that contain antibody index oligonucleotide tag sequences. Additive primers may be added to the cDNA PCR to increase yield of antibody DNA labels. Antibody index oligonucleotide tags may be identified by qPCR as a control check. The PCR products can then be purified and sequenced using known sequencing techniques (e.g., Illumina sequencing).

In certain methods and systems, specific antibodies are conjugated directly to the template particles in order to allow for selective EV or particle capture based on surface antigen identity. In such cases, a library of specific labeled template particles can be incubated with a population of EV, and the type, class or subclass of captured EV may then be determined by barcode elements that identify the antigen capture probe on the template particle.

Other capture probes may also be included on template particles depending on the desired application, including small molecule drugs to select for particular receptors, RNA derived aptamers, or DNA sequences for specific hybridization of targeted DNA sequences.

In certain aspects, methods of the invention include combining template particles with target EVs in a first fluid, adding a second fluid to the first fluid, shearing the fluids to generate a plurality of monodisperse droplets simultaneously that contain a single one of the template particles and a single one of the target EVs, in which the template particles preferably include one or more oligos useful in template switching oligo (TSO) embodiments. The method preferably also includes lysing each of the single target EVs contained within the monodisperse droplets to release a plurality of distinct mRNA molecules; and quantifying the plurality of distinct mRNA molecules by, for example, using template switching PCR (TS-PCR), as discussed in U.S. Pat. No. 5,962,272, which is incorporated herein by reference. TS-PCR is a method of reverse transcription and polymerase chain reaction (PCR) amplification that relies on a natural PCR primer sequence at the polyadenylation site, also known as the poly(A) tail, and adds a second primer through the activity of murine leukemia virus reverse transcriptase. This method permits reading full cDNA sequences and can deliver high yield from single sources, even single EVs that contain 10 to 30 picograms of mRNA.

TS-PCR generally relies on the intrinsic properties of Moloney murine leukemia virus (MMLV) reverse transcriptase and the use of a unique TSO. During first-strand synthesis, upon reaching the 5′ end of the mRNA template, the terminal transferase activity of the MMLV reverse transcriptase adds a few additional nucleotides (mostly deoxycytidine) to the 3′ end of the newly synthesized cDNA strand. These bases may function as a TSO-anchoring site. After base pairing between the TSO and the appended deoxycytidine stretch, the reverse transcriptase “switches” template strands, from EV RNA to the TSO, and continues replication to the 5′ end of the TSO. By doing so, the resulting cDNA contains the complete 5′ end of the transcript, and universal sequences of choice are added to the reverse transcription product. This approach makes it possible to efficiently amplify the entire full-length transcript pool in a completely sequence-independent manner.

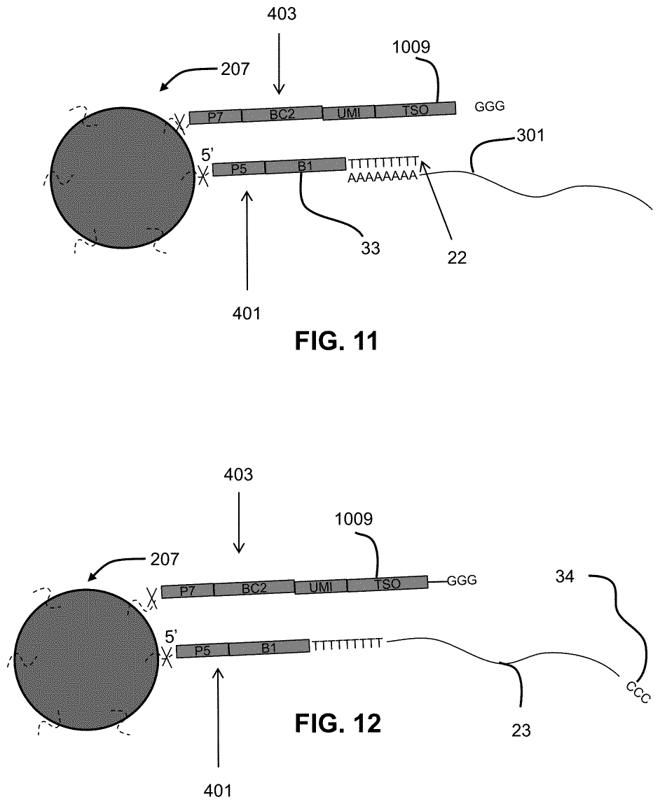

FIG. 11 illustrates the capture of mRNA 301 according to TSO embodiments. The TSO 1009 is an oligo that hybridizes to untemplated C nucleotides added by the reverse transcriptase during reverse transcription. The TSO may add, for example, a common 5′ sequence to full length cDNA that is used for downstream cDNA amplification. Shown, is a template particle 207 that comprises a first capture probe 401, and a second capture probe 403. The first capture probe 401 preferably comprises, from 5′ end to 3′ end, a linker region to allow a covalent bond with the template particle 207, a P5 nucleotide sequence region comprising a universal primer nucleotide sequence, at least one barcode 33, and a capture nucleotide sequence 22 comprising a poly T nucleotide sequence. The second capture probe 403 preferably includes a TSO 1009, a UMI, a second barcode, a P7 nucleotide sequence region comprising a universal primer nucleotide sequence. A released nucleic acid, i.e., mRNA molecule 301 comprising a poly A sequence attaches to the first capture probe's 401 poly T sequence 22 via complementary base pairing. Following the hybridization of the mRNA molecule 301 and the capture probe 401, TS-PCR is performed using a reverse transcriptase, i.e., murine leukemia virus reverse transcriptase, to synthesize cDNA and thereby create a first strand. During TS-PCR amplification, upon reaching the 5′ end of the mRNA template, the terminal transferase activity of the reverse transcriptase adds a few additional nucleotides (mostly deoxycytidine), to the 3′ end of the nascent first strand.

FIG. 12 shows a first strand 23 following TS-PCR amplification. The first strand 23 includes additional nucleotides that may function as a TSO-anchoring site 34. The TSO-anchoring site 34 may hybridize with the TSO 1009, after base pairing between the TSO and the TSO-anchoring site 34, the reverse transcriptase “switches” template strands, from EV RNA to the TSO, and continues replication to the 5′ end of the TSO. By doing so, the resulting cDNA contains the complete 5′ end of the transcript, and sequences from the second capture probe 403.

After synthesis of the first strand 23, the first strand 23 including capture probes 401, 403, may be released either by cleaving covalent bonds attaching the capture probes 401, 403 to a surface of the template particle 207, or by dissolving the template particle 207, for example, by heat.

A person with ordinary skills in the art will appreciate that any one of the template particle embodiments, capture probes, primer probes, second strand primers, universal amplification primers, barcodes, UMIs, TSOs, and methods thereof described in any one of the embodiments of the presently disclosed targeted library preparation method may be used in a different combination, or embodiment, of the present method. For example, any one of the presently described second strand primers, or primer probe, may be used to prime any one of the presently disclosed first strands to allow for a DNA synthesis reaction to generate an amplicon.

In preferred embodiments, quantifying released mRNA comprises sequencing, which may be performed by methods known in the art. For example, see, generally, Quail, et al., 2012, A tale of three next generation sequencing platforms: comparison of Ion Torrent, Pacific Biosciences and Illumina MiSeq sequencers, BMC Genomics 13:341. Nucleic acid sequencing techniques include classic dideoxy sequencing reactions (Sanger method) using labeled terminators or primers and gel separation in slab or capillary, or preferably, next generation sequencing methods. For example, sequencing may be performed according to technologies described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and U.S. Pat. No. 6,911,345, each incorporated by reference.

The conventional pipeline for processing sequencing data includes generating FASTQ-format files that contain reads sequenced from a next generation sequencing platform, aligning these reads to an annotated reference genome, and quantifying expression of genes. These steps are routinely performed using known computer algorithms, which a person skilled in the art will recognize can be used for executing steps of the present invention. For example, see Kukurba, Cold Spring Harb Protoc, 2015 (11):951-969, incorporated by reference.

After obtaining expression profiles from single EV, the expression profiles can be analyzed by, for example, comparing the profiles with reference or control profiles to ascertain information about the single target EVs.

In one aspect, methods and systems of the invention provide a method for identifying an EV of a particular subclass from a heterogeneous EV population.

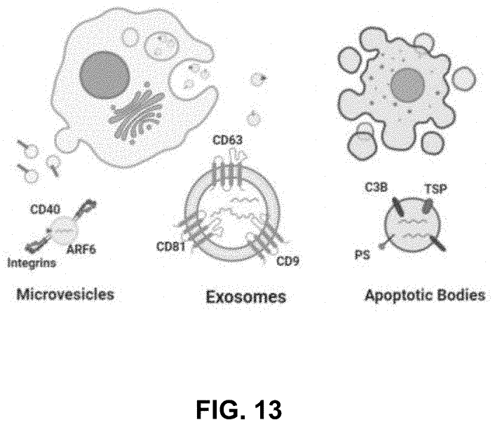

FIG. 13 provides a schematic showing a heterogeneous EV population with just a fraction of the diversity of known EV types and proteins that are specifically associated with each type.

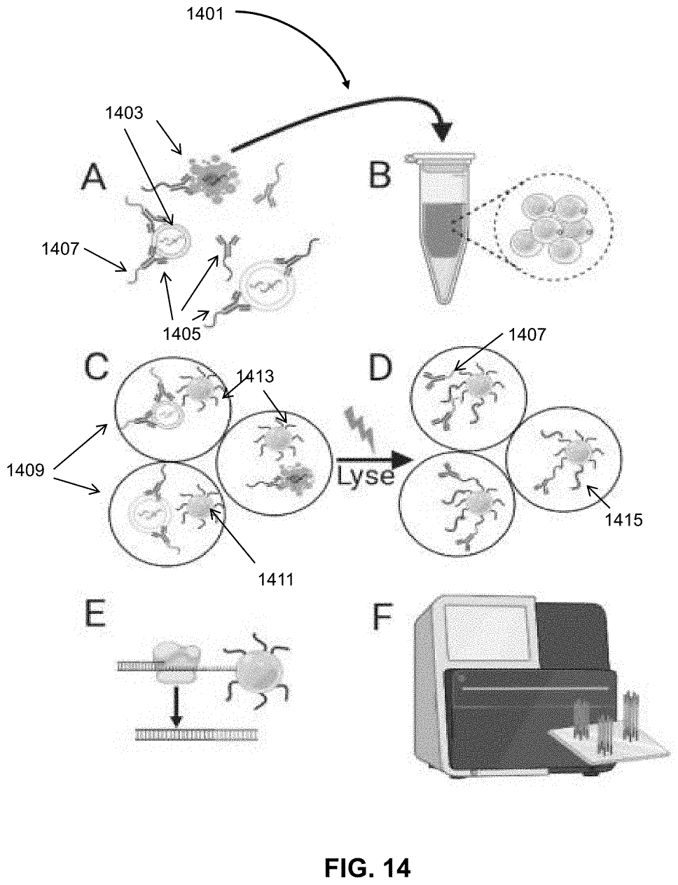

FIGS. 14A-14F provide an overview of a method 1401 for identifying an EV of a particular subclass from a heterogenous EV population.

In FIG. 14A, a population of heterogenous EVs 1403 is incubated in an aqueous fluid with target-specific antibodies 1405 that each bind to a different EV-associated target protein. Each antibody 1405 is conjugated to an index oligonucleotide tag 1407, which includes a barcode identifying the antibody, and by extension, the protein to which it binds.

In FIG. 14B, the antibody-bound EVs 1403 are sheared with a partitioning fluid, such as oil, and template particles, to create monodisperse water-in-oil droplets.

FIG. 14C shows the resulting water-in-oil droplets 1409 that each contain one template particle 1411 and one EV 1403 bound to one or more of the antibodies 1405. Each template particle includes a plurality of capture oligonucleotides 1413. The EVs inside each partition are lysed to release mRNA.

FIG. 14D shows the released mRNA 1415 and index oligonucleotides 1407 from the antibodies 1405 captured by the capture oligonucleotides 1413 on the template particles 1411.

FIG. 14E shows that the captured mRNA 1415 and index oligonucleotides 1407 are reverse transcribed, if required, amplified, and prepared as sequencing libraries.

FIG. 14F shows sequencing of the sequencing libraries to produce sequence reads.

The resulting sequence reads include barcodes from the index oligonucleotide, which are used to ascertain whether a particular EV associated protein was included in a particular droplet. Quantification and/or identification of these index oligonucleotides can provide the identity of a particular EV type captured in a particular partition, based on known associations of EV types and certain EV-associated proteins. Similarly, information from the sequenced cDNA library of mRNAs can be used to identify certain EV types included in a sample.

In certain aspects, the method 1401 includes contacting isolating a plurality of single target EVs from the heterogeneous EV population by combining the heterogeneous EVs with a plurality of template particles in a first fluid, adding a second fluid that is immiscible with the first fluid, and shearing the fluids to generate an emulsion comprising monodisperse droplets that each contain a single target EV and a single template particle. Antibody conjugates may also be included before emulsion generation such that isolation of target EVs in the heterogeneous EV population will also isolate target-protein-bound antibody conjugates for incorporation in the monodisperse droplets. Methods may further include releasing one or more biological component, e.g., a nucleic acid, from each of the single target EVs contained within the monodisperse droplets and quantifying the plurality of mRNA molecules along with identifying and quantifying the expressed target proteins based on the presence and amount of antibody conjugate labels sequenced. Quantifying may include generating a plurality of amplicons of the mRNA molecules wherein each of the amplicons comprise a barcode or index sequence that is unique to the EV from which the mRNA molecule was derived. In some instances, methods may include sequencing the plurality of barcoded amplicons by, for example, next-generation sequencing methods to generate sequence reads for each of the amplicons. Methods may further include processing the sequence reads associated with single EVs of the heterogeneous EV population to generate expression profiles for each of the single EVs and using the data by, for example, performing a gene clustering analysis to identify one or more EV subclasses. In certain aspects, other nucleic acids (e.g., genomic DNA and other RNA species) are released, captured, and sequenced using such a method. For example, EVs are known to contain several types of RNA species of clinical importance, such as micro RNAs, snRNAs, tRNAs, ncRNAs, vault RNAs, and the like. These RNAs can be captured, reverse transcribed, and sequence in accordance with the methods of the disclosure.

In certain aspects, the type of EV is determined for a plurality of EVs in a sample. The quantities of certain types of in a sample EVs can be determined. In certain aspects, these quantities can be compared, e.g., using a ratio. The identities and quantities of certain types of EVs can be used, for example, to diagnose or assess a certain pathology in a subject.

EVs represent a novel target for non-invasive diagnostics in cancer, neurological disease, and a variety of other disorders. The nucleic acid payload in EVs is protected by a lipid bilayer enabling analysis of otherwise labile RNA signatures used in alternative analysis methods from liquid biopsies. The quantity of EVs in blood has been shown to be enhanced in several disease states, and apoptotic bodies are an indicator of advanced disease with increased cell death.

Recent research has demonstrated important roles for EVs in a variety of diseases. EVs are implicated as intracellular signaling vehicles in neurological disorders including Alzheimer's, Parkinson's, Huntington's, and traumatic neuronal injuries such as Chronic Traumatic Encephalopathy and stroke. EVs are also potential biomarkers for several cancers, including colon cancer, ovarian, lung, and glioblastoma. Apoptotic bodies have been shown to induce procoagulation activity due to enriched tissue factor, and may assist in inducing an anti-cancer immunogenic response. Exosomes can stimulate proliferation of cancer cells, mediate activation of epithelial-mesenchymal transition, and induce pre-metastatic niche formation. The role of EVs in melanoma pathology has been well described in the literature. Melanoma cells produce more microvesicles than normal melanocytes, and exosomes secreted by melanoma cells have been shown to induce dysfunction in cytotoxic T-cells important in the host immune surveillance of neoplastic cells. Overall, EVs have been shown to play a variety of roles in disease formation and progression and further analysis of these vesicles can yield new insights into diagnosis and treatment of these disorders.

The presently disclosed methods for high-throughput, single EV transcriptomic analysis would enable detection and classification of vesicles originating from rare cell types in the background of those derived from normal, healthy cells. The presently disclosed methods and systems provide the ability to analyze individual EVs and link individual EV protein markers with nucleic acid payloads, providing previously inaccessible insight into the diversity of EVs in disease, their roles in pathology, and their utility as early diagnostic tools.

Any one of the presently disclosed methods and systems can include a targeted library preparation method, in which the template particle further includes a capture moiety. In some methods and systems, the capture moiety acts to capture specific target particles, for example, specific types of EV. In certain aspects, the capture moiety includes an Acrylate-terminated hydrocarbon linker with biotin termination. In certain aspects, the capture moiety is attached to a target-specific capture element. In certain aspects, the target-specific capture element is selected from aptamers and antibodies. Embodiments of the capture moiety and methods thereof are disclosed in PCT Application Serial No. PCT/US2019/053426, incorporated herein by reference.

### EXAMPLES

### Example 1

### Establish EV Model Systems

In order to establish genetically distinct EV populations, EVs are derived from human (HEK 293T) and mouse (3T3) cell cultures, which have themselves previously been characterized by PIPseq single-cell transcriptomics. A clinically-relevant melanoma cell line, NRAS mutant A375 isogenic cells, is established. Briefly, the cells are grown per ATCC guidelines.

EVs are then isolated from the cell cultures. This includes obtaining 0.5 mL of culture supernatant that is diluted into 5 mL of PBS. EVs are purified from the resulting solution using the ExoQuick-TC ULTRA kit (System Biosciences, Palo Alto, Calif.). The kit is designed specifically to isolate a pure population of EVs from a solution for use in downstream applications and analysis.

To ensure scientific rigor, a portion of the EV preparation will be used in nanoparticle tracking analysis (NTA) or methods using ELISA. Methods for NTA include, for example, those provided in Szatanek et al., “The Methods of Choice for Extracellular Vesicles (EVs) Characterization”, Int. J. Mol. Sci. 18 (2017), which is incorporated herein by reference. These methods are used to quantify the amount of EVs proceeding on to single EV capture. Quantification of EVs can be accomplished by measuring, for example, acetylcholinesterase activity using commercially available reagents, such as EXOCET Exosome Quantitation Kit (System Biosciences). RNA content of a portion of the EVs is evaluated by Bioanalyzer assay (Agilent Technologies, Inc., Santa Clara, Calif.).

Samples of EVs are shown to have increased acetylcholinesterase activity, EV-specific protein biomarker expression (CD9 and CD81 ELISA expression assays), and characteristic RNA content when compared with unpurified cell culture medium controls.

### Example 2

### Small Scale PIPseq-EV for Single-Vesicle mRNA Sequencing

To establish the ability of the present invention to isolate and analyze individual biological components of individual EVs from a sample, pre-templated instant partition sequencing of EVs (PIPseq-EV) is carried out using a sample with a small number (2,000-3,000) of EVs.

Briefly, EVs from HEK 293, 3T3, and A375 cell lines are obtained in a purified sample. EV concentration in the samples is quantified by ELISA assay or by NTA analysis. EVs are suspended in a loading buffer, their number quantified, and the EVs diluted to a prescribed concentration. For each cell type, 2,000-3,000 EVs are incubated with a panel of target-specific antibodies conjugated to index oligonucleotides. Each antibody of the panel binds to a different EV target protein and has an index oligonucleotide with a barcode that identifies the antibody/target protein to which it binds. Individual EVs bound to target-specific antibodies are isolated in monodisperse water-in-oil droplets with a single template particle.

The isolated EVs are lysed in monodisperse droplets, and released mRNA is captured by capture oligonucleotides on the template particle in each droplet. The capture oligonucleotides include a barcode identifying the particular template particle to which it is attached. The index oligonucleotide of the antibody conjugates is likewise captured by capture oligonucleotides attached to the template particles. The released mRNA is reverse transcribed and amplified to form cDNA. Illumina compatible sequencing libraries are prepared from the cDNA and the captured index oligonucleotides from the antibody conjugates using standard molecular methods.

The cDNA library fragment length distribution is quantified by Tapestation (Agilent Technologies, Inc.) and library mass quantitated using Qubit analysis (Thermofisher).

When the method produces a sufficient library yield, sequencing is performed on an Illumina NextSeq 2000 instrument. Using proprietary analytical pipelines, the sequencing results for unique transcripts of each template particle, and by extension each isolated EV, are determined. This analysis also reveals sequencing saturation and transcript diversity from EVs derived from each of the three cell lines.

### Example 3

### Refining Computational Analysis PIPseq-EV Libraries

PIPseq-EV data analysis integrates the following general steps: (1) sequencing reads are filtered for quality, and reads that meet a quality threshold with correct barcode structures (i.e., from capture oligonucleotides and index oligonucleotides); (2) unique molecules are identified by molecule-specific barcode sequences (e.g., UMIs integrated into the capture oligonucleotides); (3) reads are clustered by common PIP template barcodes, indicating the individual molecules share a common origin from an individual EV; (4) gene identity is assigned by mapping to a reference transcriptome; and (5) EV barcodes are distinguished from background noise using open-source algorithms, such as those disclosed in Lun et al., “EmptyDrops: distinguishing cells from empty droplets in droplet-based single-cell RNA sequencing data”, Genome Biol. 20, 63 (2019), which is incorporated herein by reference.

To account for the expected lower RNA content and diversity in EVs compared to isolated cells, the calling thresholds are tuned (e.g., greater than 100 genes). To establish a robust performance of the PIPseq-EV analytical pipeline, existing data sets for PIPseq of single cells are processed by down-sampling raw sequencing data to decreasing input reads. Limiting thresholds for robust assignment of cell-associated transcripts relative to background noise are established to guide threshold requirements for PIPseq-EV datasets.

To determine appropriate EV sample loading, controlled experiments are conducted in which the ratios of PIP template particles to EVs derived from mixed human and mouse cell experiments is varied. This enables sequencing-based quantification of the collision rate (human and mouse EVs) and thus through iterative experiments, a determination of a PIP template particle:EV ratio that leads to specified collision rates (e.g., <1%) can be established. Additional control experiments are carried out to establish baseline background noise, which PIPseq-EV is carried out on paired EV sample preparations with background control samples depleted of intact EVs by ultracentrifugation and retention of the resulting supernatant. This enables comparison of EVs with paired residual vesicle-free mRNA signals in sequencing analyses.

By undertaking these steps, the performance of PIPseq-EV is evaluated on single and mixed populations of EVs. A collision <5% between mouse and human EV-derived transcripts is established. EV transcriptomic content is shown to be clearly separable from vesicle-free background mRNA.

### Example 4

### Developing Panels of Barcoded Antibodies that Differentiate EV Subtypes and Enhance PIPseq-EV

The methods for single vesicle multi-omic analyses provided herein provide the capability to identify and distinguish between vesicle types and parental cell origin based on antibody epitope profiling and tying that identity to distinct genomic payloads.

This experiment provides a characteristic epitope panel capable of distinguishing, for example, exosome, microvesicle, and apoptotic body populations, and incorporate DNA labeled antibodies into the PIPseq-EV assay in order to provide multi-omic profiling of EVs isolated from Experiments 1-3. While tetraspanins, CD63, CD9 and CD81 are commonly used as antibodies for enriching exosomes, they are not sufficient to distinguish between different classes and subclasses of EV. By adding multiple markers for each EV subtype, such as C3B and Thrombosponin for apoptotic bodies, and CD4OL and B1 integrin microvesicles, panels are created to increase the specificity for different EV populations in a sample.

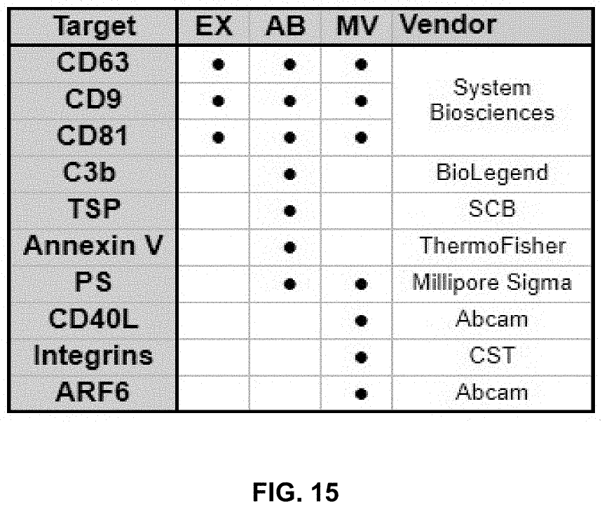

FIG. 15 provides a table showing certain target proteins, and the types of EV which they are associated with, i.e., exosomes (EX), apoptotic bodies (AB), and microvesicles (MV).

The listed antibodies are prepared, and their specific binding to certain types of EV is confirmed, for example, by using an ELISA assay. Antibodies that show the requisite, specific affinity for their target protein are modified to covalently link to an index oligonucleotide. Oligo-antibody conjugation is confirmed by positive control and their ability to bind to their target proteins is reconfirmed using bulk EV preparations by Western blot after DNA conjugation to ensure that binding epitopes are not compromised by the labeling process. As a result of these steps, a panel of at least 10 DNA-labeled antibodies is created that can be used to discriminate between classes of EVs.

### Example 5

### PIPseq-EV with Integrated Antibody Labeling

Isolated EVs are quantified and resuspended in antibody-binding buffers. The EVs are contacted with the panel of antibodies from Example 4, which each are conjugated to an antibody-specific index oligonucleotide. After incubation, unbound antibodies are removed from the labeled EV population by size exclusion chromatography using disposable columns (Zeba™ Desalting Chromatography Cartridges), and the resulting population of EVs is quantified. EVs are processed by the PIPseq-EV workflow. In the lysis and annealing steps, the DNA labels on EV bound antibodies associate with the Poly-T capture moieties on PIPs template particles. These antibody labels are transcribed along with any captured mRNA present in the individual vesicles. The resulting cDNA libraries therefore share a common vesicle barcode, provided by the PIP template particle and comprise a combination of antibody tags and mRNAs. The PIPseq-EV analysis pipeline is modified so that each of the anticipated antibody barcode sequences are whitelisted for identification. Individual populations of vesicles are classified by multiplex epitope labeling as well as transcriptomic diversity. Separation of exosomes, apoptotic bodies, and microvesicles is achieved using dimension reduction techniques (e.g. UMAP 46) using EV-subtype specific transcriptomic markers and EV-subtype specific DNA-labeled antibody markers.

### Example 6

### Expand Antibody Panel to Include Epitopes Found on Oncosomes Secreted from Melanoma Cells

The antibody panel from Example 4 is expanded to include epitopes that are specific to EVs secreted from metastatic melanoma cells, NRAS A375M. The specificity of antibodies EGFR, EPHB2, FAK, and SRC47 is tested before and after DNA label conjugation using ELISA. Once antibody specificity is confirmed, PIPseq-EV is performed on EVs from control HEK 293 epithelial cells, NRAS A375M metastatic melanoma cells, and a mixture of both. This permits an evaluation of the sensitivity of PIPseq-EV to discriminate the presence of onco-specific EVs from a healthy cell background. These steps lead to the identification of onco-specific single vesicle biomarkers in a background of non-cancerous markers.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for single cell analysis, the method comprising:
preparing a mixture comprising extracellular vesicles, target-specific antibodies linked to index oligonucleotides, and template particles comprising capture oligonucleotides;
introducing a partitioning oil to the mixture;
shearing the mixture to form a plurality of water-in-oil partitions, wherein individual extracellular vesicles are (i) isolated in one of the partitions with template particles and (ii) bound by at least one of the antibodies;
lysing the extracellular vesicles to release nucleic acid within each partition; and
analyzing the index oligonucleotides and released nucleic acid to determine one or more characteristics of the cell.

2. The method of claim 1, wherein the index oligonucleotides contain a barcode sequence that identifies a protein to which the antibody binds.

3. The method of claim 2, wherein the capture oligonucleotides of each template particle comprise a partition barcode unique to each template particle.

4. The method of claim 3, further comprising creating a sequencing library containing copies of the index oligonucleotide barcodes, the partition barcode, and the released nucleic acid.

5. The method of claim 4, further comprising sequencing the library to produce sequence reads.

6. The method of claim 5, further comprising identifying from the sequence reads proteins and nucleic acids present in the EVs.

7. The method of claim 6, further comprising using the partition barcodes in the sequence reads to identify proteins and nucleic acids of at least one individual EV.

8. The method of claim 7, further comprising using the index oligonucleotide barcodes and/or the released nucleic acid to identify an extracellular vesicle subclass of the individual EVs.

9. The method of claim 8, wherein the subclass is one of an exosome, a microvesicle, an apoptotic body, an oncosome, and an exomere.

10. The method of claim 6, wherein the released nucleic acid is RNA.

11. The method of claim 10, further comprising reverse transcribing the released RNA captured by the capture oligonucleotides to produce a cDNA library.

12. The method of claim 11, wherein the released RNA is selected from one or more of mRNA, microRNA, ncRNA, tRNA, snRNA, and vault RNA.

13. The method of claim 12, wherein the released RNA is mRNA.

14. The method of claim 7, wherein the EVs in the aqueous mixture are from a sample from a subject.

15. The method of claim 14, further comprising assessing a pathology in the subject using extracellular vesicle subclass of one or more individual EVs in the sample.

16. The method of claim 15, wherein assessing further comprises quantifying amounts of individual EVs in the sample of a particular extracellular vesicle subclass.

17. The method of claim 15, wherein assessing further comprises analyzing the nucleic acids and/or proteins identified in the EVs.

18. The method of claim 15, wherein the target-specific antibodies are a panel of target-specific antibodies, and each antibody of the panel binds to a different protein.

19. The method of claim 18, wherein panel comprises an antibody the specifically binds to a protein selected from CD63, CD9, C3b TSP, Annexin V, Phosphatidylserine, CD40L, an integrin, and ARF6.

20. The method of claim 15, wherein the pathology is cancer and the extracellular vesicle subclass is an oncosome.
